(19) United States
(12) Patent Application Publication
(10) Pub. No.: US 2026/0273488 A1
(43) Pub. Date: Sep. 17, 2026

(54) IMPROVEMENTS IN OR RELATING TO ORGANIC COMPOUNDS

(71) Applicant: GIVAUDAN SA, Vernier (CH)

(72) Inventors: Marion DENIGOT, Argenteuil (FR); Vladica BOCOKIC, Argenteuil (FR); Emilie RAGAZZINI, Argenteuil (FR); Katerina KARAGIANNI, Argenteuil (FR)

(21) Appl. No.: 19/469,496

(22) PCT Filed: Mar. 25, 2024
(86) PCT No.: PCT/EP2024/057929

§ 371 Date: Sep. 26, 2025

(30) Foreign Application Priority Data
Mar. 31, 2023 (GB) 2304823.4

(51) Int. Cl. B01J 13/14; B01J 13/08; B01J 13/10

(52) U.S. Cl. CPC B01J 13/14; B01J 13/08; B01J 13/10

(57) ABSTRACT

The present invention relates to an encapsulated composition comprising at least one core-shell microcapsule, wherein the at least one core-shell microcapsule comprises a core comprising at least one benefit agent and a shell surrounding the core, wherein the shell comprises a cross-linked-protein coacervate, wherein the cross-linker is dihydroxyacetone. The invention also relates to methods of making such a composition, to consumer products comprising such a composition and to the use of such a composition to enhance the performance of a benefit agent in a consumer product.

The present invention relates to a microcapsule composition comprising at least one core-shell microcapsule, to a method for preparing such a microcapsule composition, to a consumer product comprising such a microcapsule composition, as well as to the use of such a microcapsule composition to enhance the performance of a benefit agent in a consumer product.

### BACKGROUND OF THE INVENTION

It is known to incorporate encapsulated benefit agents in consumer products, such as household care, personal care and fabric care products. Benefit agents include for example fragrances, cosmetic agents, food ingredients, nutraceuticals, drugs and substrate enhancers.

Encapsulated benefit agents are known in the art. They may be formed by a process of coating small solid particles or liquid droplets in a thin film of shell material. Although virtually any coating material, conceptually at least, is a candidate capsule shell material, in practice for commercial and regulatory reasons, to-date, there are relatively few materials that have been used in commercial products. Capsule shell material selection is determined by a number of factors including final application, cost, availability, processing ease, and inherent barrier properties. Defining an optimal shell material for a given application can be complex since many interacting parameters determine success of a given capsule shell material.

Microcapsules that are particularly suitable for delivery of benefit agents are core-shell microcapsules, wherein the core usually comprises the benefit agent and the shell is impervious or at least partially impervious to the benefit agent. Generally, these microcapsules are employed in aqueous media and the encapsulated benefit agents are hydrophobic. A broad selection of shell materials can be used, provided the shell material is impervious or at least partially impervious to the encapsulated benefit agent.

Benefit agents are encapsulated for a variety of reasons. Microcapsules can isolate and protect such materials from external suspending media, such as consumer product bases, in which they may be incompatible or unstable. They are also used to assist in the deposition of benefit agents onto substrates, such as skin or hair, or also fabrics or hard household surfaces in case of perfume ingredients. They can also act as a means of controlling the spatio-temporal release of a benefit agent.

A wide variety of encapsulating media as well as benefit agents suitable for the preparation of encapsulated compositions has been proposed in the prior art. Such encapsulating media include synthetic resins made from polyamides, polyureas, polyurethanes, polyacrylates, melamine-derived resins, or mixtures thereof. Encapsulated benefit agent compositions are typically prepared in the form of aqueous slurries.

Consumers are increasingly concerned about using materials obtained from non-renewable sources, such as synthetic petrochemicals, as well as about the processes for manufacturing the consumer products. The “clean label” concept is one of the biggest trends of the decade. The term itself has many definitions including sustainable, naturally sourced or bio-based and biodegradable ingredients as well as minimal processing and impact on the environment. Nevertheless, it is generally difficult to use natural materials or materials derived from nature to satisfy the requirements for suitable encapsulation compositions.

Bio-based and biodegradable ingredients for customer formulations must provide a unique combination of performance and sustainability, so consumers feel confident in the safety and efficacy of these ingredients. For instance, protein-based and especially gelatin-based core-shell microcapsules are well known in the art. Gelatin-based microcapsules are conventionally obtained by a so-called coacervation process, wherein a complex between gelatin and a polysaccharide is formed at the core/water interface. Coacervation is then followed by cross-linking, in order to stabilize the shell mechanically and thermally. Cross-linking may be achieved by using a broad selection of cross-linking agents, provided the cross-linking reaction may occur at the oil/water interface, meaning in the presence of water. Cross-linking reactions that are effective in water involve, for example, reactions between available primary amine groups of gelatin with formaldehyde, di-aldehydes, and resorcinol. However, these cross-linking agents may be problematic from a regulatory or general acceptance standpoint.

Nature-derived polymers such as proteins are attractive chemical building blocks to encapsulate agents and have been explored in the recent years by the industry. Many of these materials are also biodegradable. Therefore, there is a continuous need to provide encapsulated composition comprising increased levels of natural materials with good predicted biodegradability, such as proteins. Furthermore, the processes of manufacturing the compositions should follow the “clean label” requirements, in addition to being safe, robust and cost-efficient.

### SUMMARY OF THE INVENTION

In a first aspect, the invention provides a microcapsule composition comprising at least one core-shell microcapsule, wherein at least one core-shell microcapsule comprises a core comprising at least one benefit agent and a shell surrounding the core, wherein the shell comprises a cross-linked-protein coacervate, wherein the cross-linker is dihydroxyacetone.

In a second aspect, a method for preparing the microcapsule composition as described herein is provided.

In a further aspect, it is provided the use of a microcapsule composition as described herein to enhance the performance of a benefit agent in a consumer product.

The invention further provides a consumer product comprising a microcapsule composition as described herein.

### BRIEF DESCRIPTION OF THE DRAWINGS

FIG. 1 shows an example of a microscopic image of a composition resulting from the emulsification of a fragrance in the presence of a pea protein.

FIG. 2 shows an example of a microscopic image of a composition resulting from the emulsification of fragrance in the presence of a pea protein and dihydroxyacetone.

FIG. 3 shows an example of a microscopic image of a composition resulting from the emulsification of a fragrance in the presence of canola protein.

FIG. 4 shows an example of a microscopic image of a composition resulting from the emulsification of a fragrance in the presence of a canola protein and dihydroxyacetone.

FIG. 5a shows an example of a microscopic image of a composition resulting from the emulsification of a fragrance in the presence of a canola protein, dihydroxyacetone and arginine. FIG. 5b shows an example of a SEM image of this latter composition under high vacuum.

FIG. 6 shows an example of a SEM image of a composition resulting from the emulsification of a fragrance in the presence of a canola protein, dihydroxyacetone, arginine and isophthalic dihyrazide.

FIG. 7 shows a comparison between the microscopic aspect of microcapsules prepared using canola protein, DHA cross-linker, arginine, isophthalic dihyrazide and isocyanate (left) at 2 weeks after incorporation in a liquid detergent base and the microscopic aspect of microcapsules prepared using canola protein, DHA cross-linker, arginine and isophthalic dihyrazide (right) at about 10 min after incorporation in the same liquid detergent base, at the same perfume loading.

### DEFINITIONS

The term “benefit agent” refers to any substance which, when added to a product, may improve the perception of this product by a consumer or may enhance the action of this product in an application. Examples of benefit agents include perfume/fragrance ingredients, flavor ingredients, cosmetic ingredients, bioactive agents (such as bactericides, insect repellents and pheromones), substrate enhancers (such as silicones and brighteners), enzymes (such as lipases and proteases), dyes, pigments and nutraceuticals.

The term “microcapsules” refers to capsules of sizes ranging from 0.1 μm to 500 μm.

The term “bio-based” relates to the origin of a material and refers to materials intentionally made from substances derived from living (or once-living) organisms, as opposed to petroleum-derived materials. The definition includes both natural materials, such as naturally-extracted proteins and polysaccharides, and materials that have undergone some degree of processing, such as cellulose fibers.

“Biodegradable” materials are defined as materials whose physical and chemical properties undergo deterioration and completely degrade when exposed to the environment. This property, therefore, relates to the end-of-life of the material. Bio-based materials can be biodegradable or non-degradable. Similarly, while many bio-based materials are biodegradable (e.g., starch), not all biodegradable materials are bio-based.

In context of the present invention, a “biodegradable” ingredient, or a “biodegradable” material in general, for instance a shell material, is a material which meets the pass criteria for “inherently biodegradable” and/or “readily biodegradable” in at least one OECD biodegradation study. In order to avoid any ambiguity, this means that if an ingredient passes one test but fails one or more other ones, the pass result overrules the other test results.

“Simple coacervation” refers to the formation of an interfacial layer comprising a single polyelectrolyte.

“Complex coacervation” refers to the formation of an interfacial layer comprising a mixture of at least two polyelectrolytes.

The prefix “poly” such as in “polyamine” denotes a compound with a functionality such as an amine≥2.

Polyhydrazides are reaction products of polycarboxylic acids with hydrazine.

In the present context, a moiety is a part of a molecule that is given a name because it is identified as a part of other molecules as well.

### DETAILED DESCRIPTION

Preferred and/or optional features of the invention will now be set out. Any aspect of the invention may be combined with any other aspect of the invention unless the context demands otherwise. Any of the preferred or optional features of any aspect may be combined, singly or in combination, with any aspect of the invention, as well as with any other preferred or optional features, unless the context demands otherwise.

The applicant has surprisingly and unexpectedly found that a shell or a membrane surrounding a core comprising benefit agent can be obtained by coacervation of a cross-linked-protein, wherein the cross-linker is dihydroxyacetone.

Such a shell or membrane has a higher percentage of bio-based components than those currently known in the art, giving rise to an essentially fully bio-based encapsulating material.

The proteins, dihydroxyacetone, as well as the resulting membrane or shell are also biodegradable, providing an additional benefit.

The invention, therefore, provides a microcapsule composition comprising at least one core-shell microcapsule, wherein the at least one core-shell microcapsule comprises a core comprising at least one benefit agent and a shell surrounding the core, wherein the shell comprises a cross-linked-protein coacervate, wherein the cross-linker is dihydroxyacetone.

### Benefit Agent

Suitable benefit agents to be incorporated into the core of the core-shell microcapsules of the present invention include perfume/fragrance ingredients, flavor ingredients, cosmetic ingredients, bioactive agents (such as bactericides, insect repellents and pheromones), substrate enhancers (such as silicones and brighteners), enzymes, dyes, pigments and nutraceuticals.

In one embodiment, the at least one benefit agent may be at least one fragrance ingredient. A comprehensive list of fragrance ingredients that may be encapsulated in accordance with the present invention may be found in the perfumery literature, for example “Perfume & Flavor Chemicals”, S. Arctander (Allured Publishing, 1994). Encapsulated fragrance ingredients according to the present invention preferably comprise fragrance ingredients selected from the group consisting of ACETYL ISOEUGENOL ((E)-2-methoxy-4-(prop-1-en-1-yl)phenyl acetate); ADOXAL (2,6,10-trimethylundec-9-enal); AGRUMEX (2-(tert-butyl)cyclohexyl acetate); ALDEHYDE C 10 DECYLIC (decanal); ALDEHYDE C 11 MOA (2-methyldecanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA PURE (2-methylundecanal); ALDEHYDE C 8 OCTYLIC (octanal); ALDEHYDE C 9 ISONONYLIC (3,5,5-trimethylhexanal); ALDEHYDE C 9 NONYLIC FOOD GRADE (nonanal); ALDEHYDE C 90 NONENYLIC ((E)-non-2-enal); ALDEHYDE ISO C 11 ((E)-undec-9-enal); ALDEHYDE MANDARINE ((E)-dodec-2-enal); ALLYL AMYL GLYCOLATE (prop-2-enyl 2-(3-methylbutoxy)acetate); ALLYL CAPROATE (prop-2-enyl hexanoate); ALLYL CYCLOHEXYL PROPIONATE (prop-2-enyl 3-cyclohexylpropanoate); ALLYL OENANTHATE (prop-2-enyl heptanoate); AMBER COR E1-((2-(tert-butyl)cyclohexyl)oxy)butan-2-ol, AMBERKETAL (3,8,8,11a-tetramethyldodecahydro-1H-3,5a-epoxynaphtho[2,1-c]oxepine); AMBERMAX (2-(2,2,7,7-Tetramethyltricyclo[6.2.1.0](1,6)undec-4-en-5-yl)propan-1-ol and 2-(2,2,7,7-Tetramethyltricyclo[6.2.1.0](1,6)undec-5-en-5-yl)propan-1-ol); AMBRETTOLIDE ((Z)-oxacycloheptadec-10-en-2-one); AMBROFIX ((3aR,5aS,9aS,9bR)-3a,6,6,9a-tetramethyl-2,4,5,5a,7,8,9,9b-octahydro-1H-benzo[e][1]benzofuran); AMYL BUTYRATE (pentyl butanoate); AMYL CINNAMIC ALDEHYDE ((Z)-2-benzylideneheptanal); AMYL SALICYLATE (pentyl 2-hydroxybenzoate); ANETHOLE SYNTHETIC ((E)-1-methoxy-4-(prop-1-en-1-yl)benzene); ANISYL ACETATE (4-methoxybenzyl acetate); APHERMATE (1-(3,3-dimethylcyclohexyl)ethyl formate); AUBEPINE PARA CRESOL (4-methoxybenzaldehyde); AURANTIOL ((E)-methyl 2-((7-hydroxy-3,7-dimethyloctylidene)amino)benzoate); BELAMBRE ((1R,2S,4R)-2′-isopropyl-1,7,7-trimethylspiro[bicyclo[2.2.1]heptane-2,4′-[1,3]dioxane]); BENZALDEHYDE (benzaldehyde); BENZYL ACETATE (benzyl acetate); BENZYL ACETONE (4-phenylbutan-2-one); BENZYL BENZOATE (benzyl benzoate); BENZYL SALICYLATE (benzyl 2-hydroxybenzoate); BERRYFLOR (ethyl 6-acetoxyhexanoate); BICYCLO NONALACTONE (octahydro-2H-chromen-2-one); BOISAMBRENE FORTE ((ethoxymethoxy)cyclododecane); BOISIRIS ((1S,2R,5R)-2-ethoxy-2,6,6-trimethyl-9-methylenebicyclo[3.3.1]nonane); BORNEOL CRYSTALS ((1S,2S,4S)-1,7,7-trimethylbicyclo[2.2.1]heptan-2-ol); BORNYL ACETATE ((2S,4S)-1,7,7-trimethylbicyclo[2.2.1]heptan-2-yl acetate); BOURGEONAL (3-(4-(tert-butyl)phenyl)propanal); BUTYL BUTYRO LACTATE (1-butoxy-1-oxopropan-2-yl butanoate); BUTYL CYCLOHEXYL ACETATE PARA (4-(tert-butyl)cyclohexyl acetate); BUTYL QUINOLINE SECONDARY (2-(2-methylpropyl)quinoline); CAMPHOR SYNTHETIC ((1S,4S)-1,7,7-trimethylbicyclo[2.2.1]heptan-2-one); CARVACROL (5-isopropyl-2-methylphenol); CARVONE LAEVO ((5R)-2-methyl-5-prop-1-en-2-ylcyclohex-2-en-1-one); CASHMERAN (1,1,2,3,3-pentamethyl-2,3,6,7-tetrahydro-1H-inden-4(5H)-one); CASSYRANE (5-tert-butyl-2-methyl-5-propyl-2H-furan); CEDRENE ((1S,8aR)-1,4,4,6-tetramethyl-2,3,3a,4,5,8-hexahydro-1H-5,8a-methanoazulene); CEDRYL ACETATE ((1S,6R,8aR)-1,4,4,6-tetramethyloctahydro-1H-5,8a-methanoazulen-6-yl acetate); CEDRYL METHYL ETHER ((1R,6S,8aS)-6-methoxy-1,4,4,6-tetramethyloctahydro-1H-5,8a-methanoazulene); CETONE V ((E)-1-(2,6,6-trimethylcyclohex-2-en-1-yl)hepta-1,6-dien-3-one); CINNAMIC ALCOHOL SYNTHETIC ((E)-3-phenylprop-2-en-1-ol); CINNAMIC ALDEHYDE ((2E)-3-phenylprop-2-enal); CINNAMYL ACETATE ((E)-3-phenylprop-2-en-1-yl acetate); CIS JASMONE ((Z)-3-methyl-2-(pent-2-en-1-yl)cyclopent-2-enone); CIS-3-HEXENOL ((Z)-hex-3-en-1-ol); CITRAL TECH ((E)-3,7-dimethylocta-2,6-dienal); CITRATHAL R ((Z)-1,1-diethoxy-3,7-dimethylocta-2,6-diene); CITRONELLAL (3,7-dimethyloct-6-enal); CITRONELLOL EXTRA (3,7-dimethyloct-6-en-1-ol); CITRONELLYL ACETATE (3,7-dimethyloct-6-en-1-yl acetate); CITRONELLYL FORMATE (3,7-dimethyloct-6-en-1-yl formate); CITRONELLYL NITRILE (3,7-dimethyloct-6-enenitrile); CLONAL (dodecanenitrile); CORANOL (4-cyclohexyl-2-methylbutan-2-ol); COSMONE ((Z)-3-methylcyclotetradec-5-enone); COUMARIN PURE CRYSTALS (2H-chromen-2-one); CRESYL ACETATE PARA ((4-methylphenyl) acetate); CRESYL METHYL ETHER PARA (1-methoxy-4-methylbenzene); CUMIN NITRILE (4-isopropylbenzonitrile); CYCLAL C (2,4-dimethylcyclohex-3-ene-1-carbaldehyde); CYCLAMEN ALDEHYDE EXTRA (3-(4-isopropylphenyl)-2-methylpropanal); CYCLOGALBANATE (allyl 2-(cyclohexyloxy)acetate); CYCLOHEXYL ETHYL ACETATE (2-cyclohexylethyl acetate); CYCLOHEXYL SALICYLATE (cyclohexyl 2-hydroxybenzoate); CYCLOMYRAL (8,8-dimethyl-1,2,3,4,5,6,7,8-octahydronaphthalene-2-carbaldehyde); CYMENE PARA (1-methyl-4-propan-2-ylbenzene); DAMASCENONE ((E)-1-(2,6,6-trimethylcyclohexa-1,3-dien-1-yl)but-2-en-1-one); DAMASCONE ALPHA ((E)-1-(2,6,6-trimethylcyclohex-2-en-1-yl)but-2-en-1-one); DAMASCONE DELTA (1-(2,6,6-trimethyl-1-cyclohex-3-enyl)but-2-en-1-one); DECALACTONE GAMMA (5-hexyloxolan-2-one); DECENAL-4-TRANS ((E)-dec-4-enal); DELPHONE (2-pentylcyclopentanone); DELTA-3 CARENE ((1S,6S)-3,7,7-trimethylbicyclo[4.1.0]hept-3-ene); DIHEXYL FUMARATE (dihexyl-but-2-enedioate); DIHYDRO ANETHOLE (1-methoxy-4-propylbenzene); DIHYDRO JASMONE (3-methyl-2-pentylcyclopent-2-enone); DIHYDRO MYRCENOL (2,6-dimethyloct-7-en-2-ol); DIMETHYL ANTHRANILATE (methyl 2-(methylamino)benzoate); DIMETHYL BENZYL CARBINOL (2-methyl-1-phenylpropan-2-ol); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butanoate); DIMETHYL OCTENONE (4,7-dimethyloct-6-en-3-one); DIMETOL (2,6-dimethylheptan-2-ol); DIPENTENE (1-methyl-4-(prop-1-en-2-yl)cyclohex-1-ene); DIPHENYL OXIDE (oxydibenzene); DODECALACTONE DELTA (6-heptyltetrahydro-2H-pyran-2-one); DODECALACTONE GAMMA (5-octyloxolan-2-one); DODECENAL ((E)-dodec-2-enal); DUPICAL ((E)-4-((3aS,7aS)-hexahydro-1H-4,7-methanoinden-5(6H)-ylidene)butanal); EBANOL ((E)-3-methyl-5-(2,2,3-trimethylcyclopent-3-en-1-yl)pent-4-en-2-ol); ESTERLY (ethyl cyclohexyl carboxylate); ETHYL ACETATE (ethyl acetate); ETHYL ACETOACETATE (ethyl 3-oxobutanoate); ETHYL CINNAMATE (ethyl 3-phenylprop-2-enoate); ETHYL HEXANOATE (ethyl hexanoate); ETHYL LINALOOL ((E)-3,7-dimethylnona-1,6-dien-3-ol); ETHYL LINALYL ACETATE ((Z)-3,7-dimethylnona-1,6-dien-3-yl acetate); ETHYL MALTOL (2-ethyl-3-hydroxy-4H-pyran-4-one); ETHYL METHYL-2-BUTYRATE (ethyl 2-methylbutanoate); ETHYL OCTANOATE (ethyl octanoate); ETHYL OENANTHATE (ethyl heptanoate); ETHYL PHENYL GLYCIDATE (ethyl 3-phenyloxirane-2-carboxylate); ETHYL SAFRANATE (ethyl 2,6,6-trimethylcyclohexa-1,3-diene-1-carboxylate); ETHYL VANILLIN (3-ethoxy-4-hydroxybenzaldehyde); ETHYLENE BRASSYLATE (1,4-dioxacycloheptadecane-5,17-dione); EUCALYPTOL ((1 s,4s)-1,3,3-trimethyl-2-oxabicyclo[2.2.2]octane); EUGENOL (4-allyl-2-methoxyphenol); EVERNYL (methyl 2,4-dihydroxy-3,6-dimethylbenzoate); FENCHYL ACETATE ((2S)-1,3,3-trimethylbicyclo[2.2.1]heptan-2-yl acetate); FENCHYL ALCOHOL ((1S,2R,4R)-1,3,3-trimethylbicyclo[2.2.1]heptan-2-ol); FENNALDEHYDE (3-(4-methoxyphenyl)-2-methylpropanal); FIXAMBRENE (3a,6,6,9a-tetramethyldodecahydronaphtho[2,1-b]furan); FIXOLIDE (1-(3,5,5,6,8,8-hexamethyl-5,6,7,8-tetrahydronaphthalen-2-yl)ethanone); FLORALOZONE (3-(4-ethylphenyl)-2,2-dimethylpropanal); FLORHYDRAL (3-(3-isopropylphenyl)butanal); FLORIDILE ((E)-undec-9-enenitrile); FLOROCYCLENE ((3aR,6S,7aS)-3a,4,5,6,7,7a-hexahydro-1H-4,7-methanoinden-6-yl propanoate); FLOROPAL (2,4,6-trimethyl-4-phenyl-1,3-dioxane); FLOROSA HC (tetrahydro-4-methyl-2-(2-methylpropyl)-2H-pyran-4-ol); FRESKOMENTHE (2-(sec-butyl)cyclohexanone); FRUCTONE (ethyl 2-(2-methyl-1,3-dioxolan-2-yl)acetate); FRUITATE ((3aS,4S,7R,7aS)-ethyl octahydro-1H-4,7-methanoindene-3a-carboxylate); FRUTONILE (2-methyldecanenitrile); GALBANONE PURE (1-(5,5-dimethylcyclohex-1-en-1-yl)pent-4-en-1-one); GARDENOL (1-phenylethyl acetate); GARDOCYCLENE ((3aR,6S,7aS)-3a,4,5,6,7,7a-hexahydro-1H-4,7-methanoinden-6-yl 2-methyl propanoate); GERANIOL ((E)-3,7-dimethylocta-2,6-dien-1-ol); GERANYL ACETATE ((E)-3,7-dimethylocta-2,6-dien-1-yl acetate); GERANYL CROTONATE ((E)-3,7-dimethylocta-2,6-dien-1-yl but-2-enoate); GERANYL ISOBUTYRATE ((E)-3,7-dimethylocta-2,6-dien-1-yl 2-methylpropanoate); GIVESCONE (ethyl 2-ethyl-6,6-dimethylcyclohex-2-enecarboxylate); HABANOLIDE ((E)-oxacyclohexadec-12-en-2-one); HEDIONE (methyl 3-oxo-2-pentylcyclopentaneacetate); HELIOTROPINE CRYSTALS (benzo[d][1,3]dioxole-5-carbaldehyde); HERBANATE ((2S)-ethyl 3-isopropylbicyclo[2.2.1]hept-5-ene-2-carboxylate); HEXENAL-2-TRANS ((E)-hex-2-enal); HEXENOL-3-CIS ((Z)-hex-3-en-1-ol); HEXENYL-3-CIS ACETATE ((Z)-hex-3-en-1-yl acetate); HEXENYL-3-CIS BUTYRATE ((Z)-hex-3-en-1-yl butanoate); HEXENYL-3-CIS ISOBUTYRATE ((Z)-hex-3-en-1-yl 2-methylpropanoate); HEXENYL-3-CIS SALICYLATE ((Z)-hex-3-en-1-yl 2-hydroxybenzoate); HEXYL ACETATE (hexyl acetate); HEXYL BENZOATE (hexyl benzoate); HEXYL BUTYRATE (hexyl butanoate); HEXYL CINNAMIC ALDEHYDE ((E)-2-benzylideneoctanal); HEXYL ISOBUTYRATE (hexyl 2-methylpropanoate); HEXYL SALICYLATE (hexyl 2-hydroxybenzoate); HYDROXYCITRONELLAL (7-hydroxy-3,7-dimethyloctanal); INDOFLOR (4,4a,5,9b-tetrahydroindeno[1,2-d][1,3]dioxine); INDOLE PURE (1H-indole); INDOLENE (8,8-di(1H-indol-3-yl)-2,6-dimethyloctan-2-ol); IONONE BETA ((E)-4-(2,6,6-trimethylcyclohex-1-en-1-yl)but-3-en-2-one); IRISANTHEME ((E)-3-methyl-4-(2,6,6-trimethylcyclohex-2-en-1-yl)but-3-en-2-one); IRISONE ALPHA ((E)-4-(2,6,6-trimethylcyclohex-2-en-1-yl)but-3-en-2-one); IRONE ALPHA ((E)-4-(2,5,6,6-tetramethylcyclohex-2-en-1-yl)but-3-en-2-one); ISO E SUPER (1-(2,3,8,8-tetramethyl-1,2,3,4,5,6,7,8-octahydronaphthalen-2-yl)ethanone); ISOAMYL ACETATE (3-methylbutyl acetate); ISOAMYL BUTYRATE (3-methylbutyl butanoate); ISOBUTYL METHOXY PYRAZINE (2-methylpropyl 3-methoxypyrazine); ISOCYCLOCITRAL (2,4,6-trimethylcyclohex-3-enecarbaldehyde); ISOEUGENOL ((E)-2-methoxy-4-(prop-1-en-1-yl)phenol); ISOJASMONE B 11 (2-hexylcyclopent-2-en-1-one); ISOMENTHONE DL (2-isopropyl-5-methylcyclohexanone); ISONONYL ACETATE (3,5,5-trimethylhexyl acetate); ISOPROPYL METHYL-2-BUTYRATE (isopropyl 2-methylbutanoate); ISOPROPYL QUINOLINE (6-isopropylquinoline); ISORALDEINE ((E)-3-methyl-4-(2,6,6-trimethylcyclohex-2-en-1-yl)but-3-en-2-one); JASMACYCLENE ((3aR,6S,7aS)-3a,4,5,6,7,7a-hexahydro-1H-4,7-methanoinden-6-yl acetate); JASMONE CIS ((Z)-3-methyl-2-(pent-2-en-1-yl)cyclopent-2-enone); JASMONYL (3-butyl-5-methyltetrahydro-2H-pyran-4-yl acetate); JASMOPYRANE FORTE (3-pentyltetrahydro-2H-pyran-4-yl acetate); JAVANOL ((1-methyl-2-((1,2,2-trimethylbicyclo[3.1.0]hexan-3-yl)methyl)cyclopropyl)methanol); KOAVONE ((Z)-3,4,5,6,6-pentamethylhept-3-en-2-one); LAITONE (8-isopropyl-1-oxaspiro[4.5]decan-2-one); LEAF ACETAL ((Z)-1-(1-ethoxyethoxy)hex-3-ene); LEMONILE ((2E,6Z)-3,7-dimethylnona-2,6-dienenitrile); LIFFAROME ((Z)-hex-3-en-1-yl methyl carbonate); LILIAL (3-(4-(tert-butyl)phenyl)-2-methylpropanal); LINALOOL (3,7-dimethylocta-1,6-dien-3-ol); LINALOOL OXIDE (2-(5-methyl-5-vinyltetrahydrofuran-2-yl)propan-2-ol); LINALYL ACETATE (3,7-dimethylocta-1,6-dien-3-yl acetate); MAHONIAL ((4E)-9-hydroxy-5,9-dimethyl-4-decenal); MALTOL (3-hydroxy-2-methyl-4H-pyran-4-one); MALTYL ISOBUTYRATE (2-methyl-4-oxo-4H-pyran-3-yl 2-methylpropanoate); MANZANATE (ethyl 2-methylpentanoate); MAYOL ((4-isopropylcyclohexyl)methanol); MEFROSOL (3-methyl-5-phenylpentan-1-ol); MELONAL (2,6-dimethylhept-5-enal); MERCAPTO-8-METHANE-3-ONE (mercapto-para-menthan-3-one); METHYL ANTHRANILATE (methyl 2-aminobenzoate); METHYL BENZOATE (methyl benzoate); METHYL CEDRYL KETONE (1-((1S,8aS)-1,4,4,6-tetramethyl-2,3,3a,4,5,8-hexahydro-1H-5,8a-methanoazulen-7-yl)ethanone); METHYL CINNAMATE (methyl 3-phenylprop-2-enoate); METHYL DIANTILIS (2-ethoxy-4-(methoxymethyl)phenol); METHYL DIHYDRO ISOJASMONATE (methyl 2-hexyl-3-oxocyclopentane-1-carboxylate); METHYL HEPTENONE PURE (6-methylhept-5-en-2-one); METHYL LAITONE (8-methyl-1-oxaspiro[4.5]decan-2-one); METHYL NONYL KETONE (undecan-2-one); METHYL OCTYNE CARBONATE (methyl non-2-ynoate); METHYL PAMPLEMOUSSE (6,6-dimethoxy-2,5,5-trimethylhex-2-ene); METHYL SALICYLATE (methyl 2-hydroxybenzoate); MUSCENONE ((Z)-3-methylcyclopentadec-5-enone); MYRALDENE (4-(4-methylpent-3-en-1-yl)cyclohex-3-enecarbaldehyde); MYRCENE (7-methyl-3-methyleneocta-1,6-diene); MYSTIKAL (2-methylundecanoic acid); NECTARYL (2-(2-(4-methylcyclohex-3-en-1-yl)propyl)cyclopentanone); NEOBERGAMATE FORTE (2-methyl-6-methyleneoct-7-en-2-yl acetate); NEOCASPIRENE EXTRA (10-isopropyl-2,7-dimethyl-1l-oxaspiro[4.5]deca-3,6-diene); NEOFOLIONE ((E)-methyl non-2-enoate); NEROLEX ((2Z)-3,7-dimethylocta-2,6-dien-1-ol); NEROLIDOL ((Z)-3,7,11-trimethyldodeca-1,6,10-trien-3-ol); NEROLIDYLE ((Z)-3,7,11-trimethyldodeca-1,6,10-trien-3-yl acetate); NEROLINE CRYSTALS (2-ethoxynaphthalene); NEROLIONE (1-(3-methylbenzofuran-2-yl)ethanone); NERYL ACETATE ((Z)-3,7-dimethylocta-2,6-dien-1-yl acetate); NIRVANOLIDE ((E)-13-methyloxacyclopentadec-10-en-2-one); NONADIENAL ((2E,6Z)-nona-2,6-dienal); NONADIENOL-2,6 ((2Z,6E)-2,6-nonadien-1-ol); NONADYL (6,8-dimethylnonan-2-ol); NONALACTONE GAMMA (5-pentyloxolan-2-one); NONENAL-6-CIS ((Z)-non-6-enal); NONENOL-6-CIS ((Z)-non-6-en-1-ol); NOPYL ACETATE (2-(6,6-dimethylbicyclo[3.1.1]hept-2-en-2-yl)ethyl acetate); NYMPHEAL (3-(4-(2-methylpropyl)-2-methylphenyl)propanal); OCTALACTONE DELTA (6-propyltetrahydro-2H-pyran-2-one); METHYL HEXYL KETONE (octan-2-one); ORANGER CRYSTALS (1-(2-naphtalenyl)-ethanone); ORIVONE (4-(tert-pentyl)cyclohexanone); PANDANOL ((2-methoxyethyl)benzene); PARA TERT BUTYL CYCLOHEXYL ACETATE (4-(tert-butyl)cyclohexyl acetate); PARADISAMIDE (2-ethyl-N-methyl-N-(m-tolyl)butanamide); PEACH PURE (5-heptyldihydrofuran-2(3H)-one); PELARGENE (2-methyl-4-methylene-6-phenyltetrahydro-2H-pyran); PELARGOL (3,7-dimethyloctan-1-ol); PEONILE (2-cyclohexylidene-2-phenylacetonitrile); PETALIA (2-cyclohexylidene-2-(o-tolyl)acetonitrile); PHARAONE (2-cyclohexylhepta-1,6-dien-3-one); PHENOXY ETHYL ISOBUTYRATE (2-(phenoxy)ethyl 2-methylpropanoate); PHENYL ACETALDEHYDE (2-phenyl-ethanal); PHENYL ETHYL ACETATE (2-phenylethyl acetate); PHENYL ETHYL ALCOHOL (2-phenylethanol); PHENYL ETHYL ISOBUTYRATE (2-phenylethyl 2-methylpropanoate); PHENYL ETHYL PHENYL ACETATE (2-phenylethyl 2-phenylacetate); PHENYL PROPYL ALCOHOL (3-phenylpropan-1-ol); PINENE ALPHA (2,6,6-trimethylbicyclo[3.1.1]hept-2-ene); PINENE BETA (6,6-dimethyl-2-methylenebicyclo[3.1.1]heptane); PINOACETALDEHYDE (3-(6,6-dimethylbicyclo[3.1.1]hept-2-en-2-yl)propanal); PIVAROSE (2,2-dimethyl-2-pheylethyl propanoate); POMAROSE ((2E,5E)-5,6,7-trimethylocta-2,5-dien-4-one); POMELOL (2,4,7-Trimethyl-6-octen-1-ol); PRECYCLEMONE B (1-methyl-4-(4-methylpent-3-en-1-yl)cyclohex-3-enecarbaldehyde); PRENYL ACETATE (3-methylbut-2-en-1-yl acetate); PRUNOLIDE (5-pentyldihydrofuran-2(3H)-one); RADJANOL SUPER ((E)-2-ethyl-4-(2,2,3-trimethylcyclopent-3-en-1-yl)but-2-en-1-ol); RASPBERRY KETONE (4-(4-hydroxyphenyl)butan-2-one); RHUBAFURAN (2,4-dimethyl-4-phenyltetrahydrofuran); ROSACETOL (2,2,2-trichloro-1-phenylethyl acetate); ROSALVA (dec-9-en-1-ol); ROSE OXIDE (4-methyl-2-(2-methylprop-1-en-1-yl)tetrahydro-2H-pyran); ROSE OXIDE CO (4-methyl-2-(2-methylprop-1-en-1-yl)tetrahydro-2H-pyran); ROSYFOLIA (1-methyl-2-(5-methylhex-4-en-2-yl)cyclopropylmethanol); ROSYRANE SUPER (4-methyl-2-phenyl-3,6-dihydro-2H-pyran); SAFRALEINE (2,3,3-trimethyl-1-indanone); SAFRANAL (2,6,6-trimethylcyclohexa-1,3-dienecarbaldehyde); SANDALORE EXTRA (3-methyl-5-(2,2,3-trimethylcyclopent-3-en-1-yl)pentan-2-ol); SCENTAURUS CLEAN (ethyl (Z)-2-acetyl-4-methyltridec-2-enoate); SCENTAURUS JUICY (4-(dodecylthio)-4-methylpentan-2-one); SERENOLIDE (2-(1-(3,3-dimethylcyclohexyl)ethoxy)-2-methylpropyl cyclopropanecarboxylate); SILVANONE SUPRA (cyclopentadecanone, hexadecanolide); SILVIAL (2-methyl-3-[4-(2-methylpropyl)phenyl]propanal); SPIROGALBANONE (1-(spiro[4.5]dec-6-en-7-yl)pent-4-en-1-one); STEMONE ((E)-5-methylheptan-3-one oxime); STYRALLYL ACETATE (1-phenylethyl acetate); SUPER MUGUET ((E)-6-ethyl-3-methyloct-6-en-1-ol); SYLKOLIDE ((E)-2-((3,5-dimethylhex-3-en-2-yl)oxy)-2-methylpropyl cyclopropanecarboxylate); TERPINENE ALPHA (1-methyl-4-propan-2-ylcyclohexa-1,3-diene); TERPINENE GAMMA (1-methyl-4-propan-2-ylcyclohexa-1,4-diene); TERPINEOL (2-(4-methylcyclohex-3-en-1-yl)propan-2-ol); TERPINEOL ALPHA (2-(4-methyl-1-cyclohex-3-enyl)propan-2-ol); TERPINEOL PURE (2-(4-methylcyclohex-3-en-1-yl)propan-2-ol); TERPINOLENE (1-methyl-4-(propan-2-ylidene)cyclohex-1-ene); TERPINYL ACETATE (2-(4-methyl-1-cyclohex-3-enyl)propan-2-yl acetate); TETRAHYDRO LINALOOL (3,7-dimethyloctan-3-ol); TETRAHYDRO MYRCENOL (2,6-dimethyloctan-2-ol); THIBETOLIDE (oxacyclohexadecan-2-one); THYMOL (2-isopropyl-5-methylphenol); TOSCANOL (1-(cyclopropylmethyl)-4-methoxybenzene); TRICYCLAL (2,4-dimethylcyclohex-3-enecarbaldehyde); TRIDECENE-2-NITRILE ((E)-tridec-2-enenitrile); TRIFERNAL (3-phenylbutanal); TROPIONAL (3-(benzo[d][1,3]dioxol-5-yl)-2-methylpropanal); TROPIONAL (3-(benzo[d][1,3]dioxol-5-yl)-2-methylpropanal); UNDECATRIENE ((3E,5Z)-undeca-1,3,5-triene); UNDECAVERTOL ((E)-4-methyldec-3-en-5-ol); VANILLIN (4-hydroxy-3-methoxybenzaldehyde); VELOUTONE (2,2,5-trimethyl-5-pentylcyclopentanone); VELVIONE ((Z)-cyclohexadec-5-enone); VIOLET NITRILE ((2E,6Z)-nona-2,6-dienenitrile); YARA YARA (2-methoxynaphtalene); ZINARINE (2-(2,4-dimethylcyclohexyl)pyridine; BOIS CEDRE ESS CHINE (cedar wood oil); EUCALYPTUS GLOBULUS ESS CHINA (eucalyptus oil); GALBANUM ESS (galbanum oil); GIROFLE FEUILLES ESS RECT MADAGASCAR (clove oil); LAVANDIN GROSSO OIL FRANCE ORPUR (lavandin oil); MANDARIN OIL WASHED COSMOS (mandarin oil); ORANGE TERPENES (orange terpenes); PATCHOULI ESS INDONESIE (patchouli oil); and YLANG ECO ESSENCE (ylang oil). These fragrance ingredients are particularly suitable for obtaining stable and performing microcapsules, owing to their favorable lipophilicity and olfactive performance.

In particularly preferred embodiments of the present invention, more than 75%, preferably more than 80%, even more preferably more than 85%, even still more preferably more than 90%, even yet still more preferably more than 95%, of the fragrance ingredients are biodegradable and selected from ACETYL ISOEUGENOL ((E)-2-methoxy-4-(prop-1-en-1-yl)phenyl acetate); ADOXAL (2,6,10-trimethylundec-9-enal); AGRUMEX (2-(tert-butyl)cyclohexyl acetate); ALDEHYDE C 10 DECYLIC (decanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA (2-methylundecanal); ALDEHYDE C 8 OCTYLIC (octanal); CYCLAMEN ALDEHYDE EXTRA (3-(4-isopropylphenyl)-2-methylpropanal); ALDEHYDE ISO C 11 ((E)-undec-9-enal); ALLYL AMYL GLYCOLATE (prop-2-enyl 2-(3-methylbutoxy)acetate); ALLYL CYCLOHEXYL PROPIONATE (prop-2-enyl 3-cyclohexylpropanoate); ALLYL OENANTHATE (prop-2-enyl heptanoate); AMBRETTOLIDE ((Z)-oxacycloheptadec-10-en-2-one); AMBROFIX ((3aR,5aS,9aS,9bR)-3a,6,6,9a-tetramethyl-2,4,5,5a,7,8,9,9b-octahydro-1H-benzo[e][1]benzofuran); AMYL SALICYLATE (pentyl 2-hydroxybenzoate); AUBEPINE PARA CRESOL (4-methoxybenzaldehyde); BENZYL ACETATE (benzyl acetate); BENZYL SALICYLATE (benzyl 2-hydroxybenzoate); BORNYL ACETATE ((2S,4S)-1,7,7-trimethylbicyclo[2.2.1]heptan-2-yl acetate); CARVACROL (5-isopropyl-2-methylphenol); CEDRENE ((1S,8aR)-1,4,4,6-tetramethyl-2,3,3a,4,5,8-hexahydro-1H-5,8a-methanoazulene); CEDRYL ACETATE ((1S,6R,8aR)-1,4,4,6-tetramethyloctahydro-1H-5,8a-methanoazulen-6-yl acetate); CEDRYL METHYL ETHER ((1R,6S,8aS)-6-methoxy-1,4,4,6-tetramethyloctahydro-1H-5,8a-methanoazulene); CITRAL ((E)-3,7-dimethylocta-2,6-dienal); CITRONELLOL (3,7-dimethyloct-6-en-1-ol); CITRONELLYL ACETATE (3,7-dimethyloct-6-en-1-yl acetate); COSMONE ((Z)-3-methylcyclotetradec-5-enone); CRESYL METHYL ETHER PARA (1-methoxy-4-methylbenzene); CYCLOHEXYL ETHYL ACETATE (2-cyclohexylethyl acetate); CYCLOHEXYL SALICYLATE (cyclohexyl 2-hydroxybenzoate); DAMASCENONE ((E)-1-(2,6,6-trimethylcyclohexa-1,3-dien-1-yl)but-2-en-1-one); DAMASCONE ALPHA ((E)-1-(2,6,6-trimethylcyclohex-2-en-1-yl)but-2-en-1-one); DECALACTONE GAMMA (5-hexyloxolan-2-one); DECENAL-4-TRANS ((E)-dec-4-enal); DIHYDRO MYRCENOL (2,6-dimethyloct-7-en-2-ol); DIPHENYL OXIDE (oxydibenzene); DIHYDRO ANETHOLE (1-methoxy-4-propylbenzene); DIHYDRO JASMONE (3-methyl-2-pentylcyclopent-2-enone); DIMETHYL ANTHRANILATE (methyl 2-(methylamino)benzoate); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butanoate); DIMETOL (2,6-dimethylheptan-2-ol); DODECALACTONE DELTA (6-heptyltetrahydro-2H-pyran-2-one); DODECALACTONE GAMMA (5-octyloxolan-2-one); DODECENAL ((E)-dodec-2-enal); EBANOL ((E)-3-methyl-5-(2,2,3-trimethylcyclopent-3-en-1-yl)pent-4-en-2-ol); ETHYL HEXANOATE (ethyl hexanoate); ETHYL METHYL-2-BUTYRATE (ethyl 2-methyl butyrate); ETHYL MALTOL (2-ethyl-3-hydroxy-4H-pyran-4-one); ETHYL OENANTHATE (ethyl heptanoate); ETHYL VANILLIN (3-ethoxy-4-hydroxybenzaldehyde); ETHYLENE BRASSYLATE (1,4-dioxacycloheptadecane-5,17-dione); EUCALYPTOL ((1s,4s)-1,3,3-trimethyl-2-oxabicyclo[2.2.2]octane); EUGENOL (4-allyl-2-methoxyphenol); EVERNYL (methyl 2,4-dihydroxy-3,6-dimethylbenzoate); FIXAMBRENE (3a,6,6,9a-tetramethyldodecahydronaphtho[2,1-b]furan); FLORHYDRAL (3-(3-isopropylphenyl)butanal); FLORIDILE ((E)-undec-9-enenitrile); GALBANONE PURE (1-(5,5-dimethylcyclohex-1-en-1-yl)pent-4-en-1-one); GARDENOL (1-phenylethyl acetate); GERANIOL ((E)-3,7-dimethylocta-2,6-dien-1-ol); GERANYL ACETATE ((E)-3,7-dimethylocta-2,6-dien-1-yl acetate); HABANOLIDE ((E)-oxacyclohexadec-12-en-2-one); HEDIONE (methyl 3-oxo-2-pentylcyclopentaneacetate); HEXENAL-2-TRANS ((E)-hex-2-enal); HEXENOL-3-CIS ((Z)-hex-3-en-1-ol); HEXENYL-3-CIS ACETATE ((Z)-hex-3-en-1-yl acetate); HEXENYL-3-CIS SALICYLATE ((Z)-hex-3-en-1-yl 2-hydroxybenzoate); HEXYL ACETATE (hexyl acetate); INDOLENE (8,8-di(1H-indol-3-yl)-2,6-dimethyloctan-2-ol); IONONE BETA ((E)-4-(2,6,6-trimethylcyclohex-1-en-1-yl)but-3-en-2-one); IRISANTHEME ((E)-3-methyl-4-(2,6,6-trimethylcyclohex-2-en-1-yl)but-3-en-2-one); IRISONE ALPHA ((E)-4-(2,6,6-trimethylcyclohex-2-en-1-yl)but-3-en-2-one); ISOAMYL ACETATE (3-methylbutyl acetate); ISOAMYL BUTYRATE (3-methylbutyl butanoate); ISOEUGENOL ((E)-2-methoxy-4-(prop-1-en-1-yl)phenol); ISOJASMONE B 11 (2-hexylcyclopent-2-en-1-one); ISORALDEINE ((E)-3-methyl-4-(2,6,6-trimethylcyclohex-2-en-1-yl)but-3-en-2-one); JASMONYL (3-butyl-5-methyltetrahydro-2H-pyran-4-yl acetate); LAITONE (8-isopropyl-1-oxaspiro[4.5]decan-2-one); LEMONILE ((2E,6Z)-3,7-dimethylnona-2,6-dienenitrile); LINALOOL (3,7-dimethylocta-1,6-dien-3-ol); LINALOOL OXIDE (2-(5-methyl-5-vinyltetrahydrofuran-2-yl)propan-2-ol); LINALYL ACETATE (3,7-dimethylocta-1,6-dien-3-yl acetate); MANZANATE (ethyl 2-methylpentanoate); MAYOL ((4-isopropylcyclohexyl)methanol); MEFROSOL (3-methyl-5-phenylpentan-1-ol); MELONAL (2,6-dimethylhept-5-enal); MERCAPTO-8-METHANE-3-ONE (mercapto-para-menthan-3-one); METHYL ANTHRANILATE (methyl 2-aminobenzoate); METHYL BENZOATE (methyl benzoate); METHYL DIANTILIS (2-ethoxy-4-(methoxymethyl)phenol); METHYL HEPTENONE PURE (6-methylhept-5-en-2-one); METHYL LAITONE (8-methyl-1-oxaspiro[4.5]decan-2-one); METHYL OCTYNE CARBONATE (methyl non-2-ynoate); METHYL SALICYLATE (methyl 2-hydroxybenzoate); NECTARYL (2-(2-(4-methylcyclohex-3-en-1-yl)propyl)cyclopentanone); NEOFOLIONE ((E)-methyl non-2-enoate); NEROLEX ((2Z)-3,7-dimethylocta-2,6-dien-1-ol); NEROLIDOL ((Z)-3,7,11-trimethyldodeca-1,6,10-trien-3-ol); NEROLINE CRYSTALS (2-ethoxynaphthalene); NEROLIONE (1-(3-methylbenzofuran-2-yl)ethanone); NERYL ACETATE ((Z)-3,7-dimethylocta-2,6-dien-1-yl acetate); NONADIENAL ((2E,6Z)-nona-2,6-dienal); NONENAL-6-CIS ((Z)-non-6-enal); NONENOL-6-CIS ((Z)-non-6-en-1-ol); NYMPHEAL (3-(4-(2-methylpropyl)-2-methylphenyl)propanal); OCTALACTONE DELTA (6-propyltetrahydro-2H-pyran-2-one); ORANGER CRYSTALS (1-(2-naphtalenyl)-ethanone); PARA TERT BUTYL CYCLOHEXYL ACETATE (4-(tert-butyl)cyclohexyl acetate); PEACH PURE (5-heptyldihydrofuran-2(3H)-one); PELARGOL (3,7-dimethyloctan-1-ol); PHENYL ETHYL ACETATE (2-phenylethyl acetate); PINENE ALPHA (2,6,6-trimethylbicyclo[3.1.1]hept-2-ene); PINENE BETA (6,6-dimethyl-2-methylenebicyclo[3.1.1]heptane); POMAROSE ((2E,5E)-5,6,7-trimethylocta-2,5-dien-4-one); POMELOL FF (2,4,7-Trimethyl-6-octen-1-ol); PRENYL ACETATE (3-methylbut-2-en-1-yl acetate); PRUNOLIDE (5-pentyldihydrofuran-2(3H)-one); RASPBERRY KETONE (4-(4-hydroxyphenyl)butan-2-one); ROSALVA (dec-9-en-1-ol); ROSE OXIDE CO (4-methyl-2-(2-methylprop-1-en-1-yl)tetrahydro-2H-pyran); ROSYRANE SUPER (4-methyl-2-phenyl-3,6-dihydro-2H-pyran); SAFRANAL (2,6,6-trimethylcyclohexa-1,3-dienecarbaldehyde); SCENTAURUS JUICY (4-(dodecylthio)-4-methylpentan-2-one); SILVIAL (2-methyl-3-[4-(2-methylpropyl)phenyl]propanal); STYRALLYL ACETATE (1-phenylethyl acetate); SYLKOLIDE ((E)-2-((3,5-dimethylhex-3-en-2-yl)oxy)-2-methylpropyl cyclopropanecarboxylate); TERPINENE GAMMA (1-methyl-4-propan-2-ylcyclohexa-1,4-diene); TERPINEOL (2-(4-methylcyclohex-3-en-1-yl)propan-2-ol); TERPINOLENE (1-methyl-4-(propan-2-ylidene)cyclohex-1-ene); TETRAHYDRO LINALOOL (3,7-dimethyloctan-3-ol); TOSCANOL (1-(cyclopropylmethyl)-4-methoxybenzene); TRIDECENE-2-NITRILE ((E)-tridec-2-enenitrile); TRIFERNAL (3-phenylbutanal); TROPIONAL (3-(benzo[d][1,3]dioxol-5-yl)-2-methylpropanal); UNDECAVERTOL ((E)-4-methyldec-3-en-5-ol); YARA YARA (2-methoxynaphtalene); BOIS CEDRE ESS CHINE (cedar wood oil); EUCALYPTUS GLOBULUS ESS CHINA (eucalyptus oil); GALBANUM ESS (galbanum oil); GIROFLE FEUILLES ESS RECT MADAGASCAR (clove oil); LAVANDIN GROSSO OIL FRANCE ORPUR (lavandin oil); MANDARIN OIL WASHED COSMOS (mandarin oil); ORANGE TERPENES (orange terpenes); PATCHOULI ESS INDONESIE (patchouli oil); and YLANG ECO ESSENCE (ylang oil). These ingredients have the advantage of providing microcapsules which are particularly sustainable.

The at least one benefit agent may comprise at least one fragrance precursor, meaning a material that is capable of releasing a fragrance ingredient by the means of a stimulus, such as a change of temperature, the presence of oxidants, the action of enzymes or the action of light. Such fragrance precursors are well-known to the art.

The at least one benefit agent may also comprise at least one functional cosmetic ingredient. The functional cosmetic ingredients for use in the encapsulated composition are preferably hydrophobic. Preferably, the cosmetic ingredients have a calculated octanol/water partition coefficient (ClogP) of 1.5 or more, more preferably 3 or more. Alternatively preferred, the ClogP of the cosmetic ingredient is from 2 to 7.

Particularly useful functional cosmetic ingredients may be selected from the group consisting of emollients, smoothening ingredients, hydrating ingredients, soothing and relaxing ingredients, decorative ingredients, deodorants, anti-aging ingredients, cell rejuvenating ingredients, draining ingredients, remodeling ingredients, skin levelling ingredients, preservatives, anti-oxidants, antibacterial or bacteriostatic ingredients, cleansing ingredients, lubricating ingredients, structuring ingredients, hair conditioning ingredients, whitening ingredients, texturing ingredients, softening ingredients, anti-dandruff ingredients, and exfoliating ingredients.

Particularly useful functional cosmetic ingredients include, but are not limited to hydrophobic polymers, such as alkyldimethylsiloxanes, polymethylsil-sesquioxanes, polyethylene, polyisobutylene, styrene-ethylene-styrene and styrene-butylene-styrene block copolymers, and the like; mineral oils, such as hydrogenated isoparaffins, silicone oils and the like; vegetable oils, such as argan oil, jojoba oil, aloe vera oil, and the like; fatty acids and fatty alcohols and their esters; glycolipides; phospholipides; sphingolipides, such as ceramides; sterols and steroids; terpenes, sesquiterpenes, triterpenes and their derivatives; essential oils, such as Arnica oil, Artemisia oil, Bark tree oil, Birch leaf oil, Calendula oil, Cinnamon oil, Echinacea oil, Eucalyptus oil, Ginseng oil, Jujube oil, Helianthus oil, Jasmine oil, Lavender oil, Lotus seed oil, Perilla oil, Rosmary oil, Sandal wood oil, Tea tree oil, Thyme oil, Valerian oil, Wormwood oil, Ylang Ylang oil, and Yucca oil.

In particular, the at least one functional cosmetic ingredient may be selected from the group consisting of Sandal wood oil, such as Fusanus Spicatus kernel oil; Panthenyl triacetate; Tocopheryl acetate; Tocopherol; Naringinin; Ethyl linoleate; Farnesyl acetate; Farnesol; Citronellyl methyl crotonate; and Ceramide-2 (1-Stearoiyl-C18-Sphingosine, CAS-No: 100403-19-8).

The at least one benefit agent may comprise agents which suppress or reduce malodour and its perception by adsorbing odour, agents which provide a warming or cooling effect, insect repellents or UV absorbers.

In one embodiment, the benefit agent is biodegradable.

### Protein Cross-Linking

Proteins are macromolecules that comprise one or more long chains of amino acid residues. Proteins and peptides are attractive chemical building blocks for encapsulation of benefit agents due to their biodegradability and added functionality compared to synthetic polymers.

Suitable proteins that may be used in the present invention include animal-origin proteins, vegetable-origin proteins or bioengineered proteins. In one embodiment, the protein is a vegetable-origin protein, such as proteins from cereals, proteins from high-protein plants, proteins from oleaginous plants or proteins from tubers.

The proteins comprised in the interfacial coacervate phase surrounding the droplets of core composition are cross-linked with a cross-linking agent. This leads to the formation of an encapsulating shell around the droplets, leading thereby to the formation of a multitude of microcapsules dispersed in an aqueous phase.

Cross-linking is the process of binding two protein moieties together. The cross-linking may be intra-molecular or extra-molecular, i.e. between two or more protein molecules. Cross-linking may be reversible or irreversible. Irreversible cross-linking may be obtained by employing cross-linking reagents (cross-linkers) that usually form covalent bonds. Cross-linking reagents usually contain two or more chemically reactive ends that attach themselves to the functional groups found in proteins. The amine functionality of a protein represents one reactive site amenable for cross-linking in view of obtaining a material suitable to act as a membrane or shell encapsulating a benefit agent.

Examples of suitable proteins in the present invention are canola or rapeseed protein, hemp protein, potato protein, chickpea protein, pea protein hydrolysate, pea protein, pea and rice fermented protein, mungbean protein, sunflower protein, pumpkin protein, carob protein, fava bean protein.

It is advantageous if the protein has a high content of aminoacids comprising a high number of amino groups, such as arginine, lysine or histidine, to enable a higher number of cross-linkages. In one embodiment, the protein has a high content of arginine or lysine, such as rapeseed or canola proteins, moringa proteins, carob proteins, pumpkin proteins or sunflower proteins.

In one embodiment, the protein is pea protein hydrolysate.

Generally, di-functional aldehydes such as succinaldehyde, glutaraldehyde, glyoxal, benzene-1,2-dialdehyde, benzene-1,3-dialdehyde, benzene-1,4-dialdehyde, piperazine-N,N-dialdehyde, and 2,2′-bipyridyl-5,5′-dialdehyde are regarded as effective cross-linking agents for proteins.

### Dihydroxyacetone

Dihydroxyacetone (or 1,3-dihydroxy-2-propanone) is a ketotriose consisting of acetone bearing hydroxy substituents at positions 1 and 3. It is often derived from plant sources such as sugar beets and sugar cane or from the fermentation of glycerin.

In one embodiment, the dihydroxyacetone is generated in situ from an esterified dihydroxyacetone precursor and a compound capable of cleaving at least one ester bond, as described in U.S. Pat. No. 5,693,670 A, which is incorporated herein by reference.

In one embodiment, the esterified dihydroxyacetone precursor is a compound of Formula 1

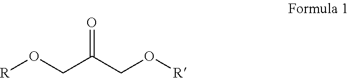

wherein R and R′ are each independently selected from the group consisting of H, a saturated or unsaturated, linear, branched or cyclic, optionally hydroxylated acyl radical having from 2 to 25 carbon atoms, provided that R and R′ are not both H.

In one embodiment, R and R′ are each independently selected from the group consisting of H, methylcarbonyl, ethylcarbonyl, benzylcarbonyl, octylcarbonyl, oleylcarbonyl, isopropyl-carbonyl, decylcarbonyl, carbonyl dioxy-2,3-propylcarbonyl, benzoyl, alkylbenzoyl and acylbenzoyl, provided that R and R′ are not both H.

The compound capable of cleaving at least one ester bond may be any cosmetically acceptable nucleophilic compound, such as an alcohol, a thiol, an amine or an anion.

In one embodiment, the compound capable of cleaving at least one ester bond is an amine, a hydroxylated amine such as, for example, 3-amino-1,2-propanediol, 2-amino-2-methyl-1,3-propanediol, 2-amino-2-methylpropanol, 2-amino-2-hydroxymethyl-1,3-propanediol, glucamine or N-methylglucamine, or an amino acid such as, for example, lysine, arginine or histidine.

The present invention shows that, surprisingly and unexpectedly, using dihydroxyacetone to cross-link a protein leads to improvement in the stability and encapsulation efficiency of a shell or a membrane comprising a cross-linked protein coacervate surrounding a core comprising benefit agent.

In one embodiment, the shell of the at least one core-shell microcapsule comprises a dihydroxyacetone-cross-linked protein coacervate, wherein the protein is pea protein or pea protein hydrolysate.

In one embodiment, the protein is sold under the tradename Roquette S85XF.

In one embodiment, the coacervate comprises a dihydroxyacetone-cross-linked pea protein and a polysaccharide, such as pectin. In one embodiment, the pectin is a high methoxylated pectin, such as a pectin sold under the tradename Roeper APA 104.

In one embodiment, the shell of the at least one core-shell microcapsule comprises a dihydroxyacetone-cross-linked protein coacervate, wherein the protein is canola protein. In one embodiment, the protein is sold under the tradename Vertis CanolaPRO.

In one embodiment, the shell of the at least one core-shell microcapsule comprises a dihydroxyacetone-cross-linked protein coacervate, wherein the protein is moringa protein.

In one embodiment, the weight ratio between the dihydroxyacetone moieties and the protein moieties in the composition is between about 2:1 to about 1:1. In one embodiment, the weight ratio between the dihydroxyacetone moieties and the protein moieties is about 1.5:1. It is believed that, in order to form as many cross-linking bonds as possible, reaction of most amino groups of the protein with dihydroxyacetone is required. The inventors therefore believe that an excess of dihydroxyacetone is required in the reaction mixture.

In one embodiment, the encapsulated composition comprises
between about 0.25 wt % to about 7.0 wt %, optionally between about 1.5 wt % to about 3.5 wt %, preferably between about 1.9 wt % to about 3.1 wt % of protein moieties; and
between about 0.5 wt % to about 7.0 wt %, optionally between about 1.5 wt % to about 3.5 wt %, preferably between about 2.7 wt % to about 3.1 wt % of dihydroxyacetone moieties.

In one embodiment, the encapsulated composition comprises about 3.0 wt % dihydroxyacetone moieties and about 2.0 wt % protein moieties. In one embodiment, the encapsulated composition comprises about 3.0 wt % dihydroxyacetone moieties and about 3.0 wt % protein moieties.

In one embodiment, the encapsulated composition comprises about 3.0 wt % dihydroxyacetone moieties and about 2.0 wt % protein moieties, wherein the protein is a pea protein, a pea protein hydrolysate or a canola protein.

### Coacervates

Coacervation involving proteins, in which the protein is subjected to either simple or complex coacervation, has been employed widely in encapsulation of benefit agent.

### Simple Coacervation

The coacervation of a single polyelectrolyte is generally induced by bringing the polyelectrolyte to its isoelectric point, meaning the point where the net charge of the polyelectrolyte is zero or close to zero. This may be achieved by changing the salt concentration or, in the case of a poly-ampholyte, such as proteins, by changing the pH of the medium.

Simple coacervation may also be induced by cross-linking a protein at the core composition/water interface.

In one embodiment, the protein is a pea protein or a pea protein hydrolysate.

In one embodiment, the protein is rapeseed or canola protein, and the coacervation process is as described in WO 2021/013710.

### Complex Coacervation

A complex coacervate can be formed between two different proteins.

This coacervation is due to the fact that the net electrical charge of the proteins may be adjusted by adjusting the pH, so that the complexation between two different ampholytic proteins is facilitated. Complexation occurs at the pH where one of the protein has an overall positive electrical charge, whereas the other protein has an overall negative charge, so that the overall electrical charge of the complex is neutral.

The phenomenon of simple or complex coacervation may be observed under a light microscope, wherein it is marked by the appearance of a ring around the core composition droplet. This ring consists of the aforementioned polyelectrolyte-rich phase that has a different refractive index than the surrounding aqueous phase.

In one embodiment the proteins forming the complex coacervate are canola protein or pea protein, each of them comprising two proteins.

In one embodiment, a cross-linked protein undergoes coacervation.

The simple or complex coacervate droplets agglomerate at the interface between the core composition and the aqueous phase. As a result, a core composition (oil phase) emulsion in water is formed, comprising a plurality of core composition droplets, each droplet being surrounded by coacervate droplets. These droplets further coalesce to form an interfacial coacervate phase surrounding the droplet. These stabilize the emulsion in that it prevents the droplets from coalesce.

In one embodiment, the protein is a canola protein or a pea protein hydrolysate.

### Second Cross-Linker

Due to the presence of unreacted dihydroxyacetone in the reaction mixture, a second cross-linker comprising more than two amino functionalities in the molecule is believed to stabilize the network of dihydroxyacetone-cross-linked protein.

Suitable second cross-linkers comprising more than two amino functionalities are selected from the groups consisting of aminoacids with more than two amino functionalities, derivatives or polymers thereof, polyhydrazides and combinations thereof.

Preferably, second cross-linkers comprising more than two amino functionalities are selected from the group consisting of arginine, polyarginine, PCA ethyl cocoyl arginate (pyrrolidone carboxylic acid ethyl cocoyl arginate), lysine, polylysine, histidine, isophthalic dihydrazide and combinations thereof.

In one embodiment, one second cross-linker comprising more than two amino functionalities is employed. In one embodiment, the second cross-linker comprising more than two amino functionalities is arginine.

In one embodiment, two or more cross-linkers comprising more than two amino functionalities are employed. In one embodiment, the second cross-linker comprising more than two amino functionalities is a combination of arginine and isophthalic dihydrazide.

In one embodiment, the shell of the at least one core-shell microcapsule comprises a dihydroxyacetone-cross-linked protein, wherein the protein is canola protein, which is further cross-linked with arginine.

In one embodiment, the shell of the at least one core-shell microcapsule comprises a dihydroxyacetone-cross-linked protein, wherein the protein is canola protein, which is further cross-linked with a combination of arginine and isophthalic dihydrazide.

### Further Cross-Linkers

In one embodiment, the shell of the at least one core-shell microcapsule comprises, in addition to a dihydroxyacetone-cross-linked protein and a second cross-linker comprising more than two amino functionalities in the molecule, one or more further cross-linkers.

In one embodiment, the further cross-linker may be selected from the group consisting of trimesoyl chloride (1,3,5-benzenetricarbonyl trichloride), a polyfunctional isocyanate, glutaraldehyde and combinations thereof.

In one embodiment, the polyfunctional isocyanate may be selected from organic isocyanates, in which an isocyanate group is bonded to an organic residue (R—N═C═O or R—NCO). In the context of the present invention, the polyfunctional isocyanate may be selected from alkyl, alicyclic, aromatic and alkylaromatic, as well as anionically modified polyfunctional isocyanates, with two or more (e.g. 3, 4, 5, etc.) isocyanate groups in a molecule.

Preferably, the polyfunctional isocyanate is an aromatic or an alkylaromatic isocyanate, the alkylaromatic polyfunctional isocyanate having preferably methylisocyanate groups attached to an aromatic ring. Both aromatic and methylisocyanate-substituted aromatic polyfunctional isocyanates have a superior reactivity compared to alkyl and alicyclic polyfunctional isocyanates. Among these, 2-ethylpropane-1,2,3-triyl tris((3-(isocyanatomethyl)phenyl)carbamate) is particularly preferred, because of its trifunctional nature that favors the formation of intermolecular cross-links and because of its intermediate reactivity that favors network homogeneity. This alkylaromatic polyfunctional isocyanate is commercially available under the trademark Takenate D-100 N, sold by Mitsui or under the trademark Desmodur® Quix175, sold by Covestro.

As an alternative to aromatic or alkylaromatic polyfunctional isocyanates, it may also be advantageous to add an anionically modified polyfunctional isocyanates, because of the ability of such polyfunctional isocyanates to react at the oil/water interface and even in the water phase close to the oil/water interface. A particularly suitable anionically modified polyfunctional isocyanate has Formula 2.

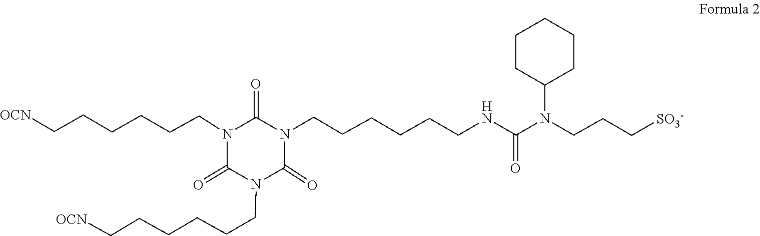

Formula 2 shows a commercially available anionically modified polyisocyanate, which is a modified isocyanurate of hexamethylene diisocyanate, sold by Covestro under the trademark Bayhydur® XP2547.

In one embodiment, the composition comprises between about 0.5 wt % to about 7 wt %, optionally between about 2.5 wt % to about 3.5 wt %, optionally about 3.0 wt % dihydroxyacetone moieties with respect to the composition. Such a level of dihydroxyacetone leads to the formation of a continuous, rigid and frangible shell around the droplets. Typically, such a shell may be broken, releasing fragments that are visible under the light microscope.

In one embodiment, the composition comprises between about 0.3 wt % to about 1.2 wt %, optionally between about 0.4 wt % to about 0.1 wt %, optionally about 0.5 wt % or about 1.0 wt % of moieties of a second cross-linker comprising more than two amino functionalities.

In one embodiment, the composition comprises between about 0.3 wt % to about 0.7 wt %, optionally between about 0.4 wt % to about 0.7 wt %, optionally about 0.5 wt % of moieties of a further isocyanate cross-linker, optionally wherein the isocyanate is an alkylaromatic polyfunctional isocyanate.

In one embodiment, the composition comprises
between about 0.25 wt % to about 7.0 wt % of protein moieties;
between about 0.5 wt % to about 7.0 wt % of dihydroxyacetone moieties;
between about 0.3 wt % to about 1.2 wt % of moieties of a second cross-linker comprising more than two amino functionalities; and optionally
between about 0.3 wt % to about 0.7 wt % of an alkylaromatic polyfunctional isocyanate.

In one embodiment, the composition comprises
about 2.0 wt % or 3.0 wt % of protein moieties;
about 3.0 wt % of dihydroxyacetone moieties;
about 0.5 wt % or about 1.0 wt % of moieties of a second cross-linker comprising more than two amino functionalities; and optionally
about 0.5 wt % of an alkylaromatic polyfunctional isocyanate.

In one embodiment, the composition comprises
about 2.0 wt % of protein moieties, wherein the protein is canola protein;
about 3.0 wt % of dihydroxyacetone moieties; and
about 0.5 wt % of arginine moieties.

In one embodiment, the composition comprises
about 2.0 wt % of protein moieties, wherein the protein is canola protein;
about 3.0 wt % of dihydroxyacetone moieties;
about 0.5 wt % of arginine moieties; and
about 0.5 wt % of isophthalic dihydrazide moieties.

In one embodiment, the composition comprises
about 2.0 wt % or 3 wt % of protein moieties, wherein the protein is canola protein or pea protein hydrolysate;
about 3.0 wt % of dihydroxyacetone moieties;
about 0.5 wt % of arginine moieties;
about 0.5 wt % of isophthalic dihydrazide moieties; and optionally
about 0.5 wt % of 2-ethylpropane-1,2,3-triyl tris((3-(isocyanatomethyl)phenyl)carbamate).

In one embodiment, the volume average size (Dv(50)) of the microcapsules is from 1 to 500 μm, optionally from 1 to 100 μm, preferably from 5 to 80 μm, even more preferably from 10 to 70 μm.

### Method

In one aspect, it is provided a method for preparing the encapsulated composition as defined herein, the method comprising the steps of:
a) providing an aqueous phase comprising a protein and dihydroxyacetone;
b) providing an oil phase comprising a benefit agent;
c) emulsifying the oil phase provided in step b) in the aqueous phase composition provided in step a);
d) inducing coacervation of the mixture obtained in step c) to obtain core composition droplets surrounded by a polymeric membrane; and
d) optionally, adding to the composition obtained in step c) a second cross-linker comprising more than two amino functionalities to obtain a microcapsule composition;
optionally wherein the oil phase in step b) comprises a further cross-linker.

The benefit agent, protein, second cross-linker comprising more than two amino functionalities and further cross-linker are as defined hereinabove.

In one embodiment, the protein is mixed with water and dihydroxyacetone and stirred at a temperature of between about 10° C. to about 35° C. for a period of time of about 8 to about 20 hours. A polysaccharide is added and stirring is increased to between about 800 rpm to about 1200 rpm and benefit agent, preferably a fragrance, is added, leading to emulsification of the composition. In one embodiment, the mixture is further stirred at a temperature between about 70° C. to about 80° C., for a period of time of between about 4 to about 8 hours to induce coacervation.

In one embodiment, the protein is mixed with water and dihydroxyacetone and stirred at a temperature of between about 10° C. to about 35° C. for a period of time of about 8 to about 20 hours. Stirring is increased to between about 800 rpm to about 1200 rpm and benefit agent, preferably a fragrance, is added, leading to emulsification of the composition.

In one embodiment, a further cross-linker, optionally a polyfunctional isocyanate, may be added in step a) and/or in step b). In one embodiment, trimesoyl chloride and/or a polyfunctional isocyanate is added in step b). In one embodiment, glutaraldehyde is added in step a). In one embodiment, a polyfuntional isocyanate is added in step b).

The polyfunctional isocyanate is as defined hereinabove.

In another aspect, a method for preparing an encapsulated composition as defined herein is provided, wherein the method comprises the steps of:
a) providing an oil phase comprising a protein and benefit agent;
b) providing an aqueous phase comprising dihydroxyacetone;
c) emulsifying the oil phase provided in step a) in the aqueous phase composition provided in step b);
d) inducing coacervation of the mixture obtained in step c) to obtain core composition droplets surrounded by a polymeric membrane;
d) optionally, adding to the composition obtained in step c) a second cross-linker comprising more than two amino functionalities to obtain a microcapsule composition, optionally wherein the oil phase in step a) comprises a further cross-linker.

The benefit agent, protein, second cross-linker comprising more than two amino functionalities and further cross-linker are as defined hereinabove.

The protein is mixed with benefit agent, preferably a fragrance, for a period of time such as of about 0.5 to about 20 hours, under moderate stirring (about 400 rpm). Thereafter, the stirring is increased to between about 800 rpm to about 1200 rpm and water, dihydroxyacetone and optionally more protein are added slowly to the oil phase.

In one embodiment, the pH of the system is adjusted to between about 8 to about 10, optionally to about 9, optionally using a Bronsted base, such as sodium hydroxide, optionally as an aqueous solution, providing conditions suitable for coacervation. In one embodiment, the mixture is further stirred at a temperature between about 70° C. to about 90° C., for a period of time of between about 4 to about 8 hours.

In one embodiment, the mixture as obtained hereinabove is subsequently gradually allowed to cool to about 25° C.

In one embodiment to the mixture as obtained hereinabove at a temperature between about 70° C. to about 90° C., it is added a second cross-linker comprising more than two amino functionalities. In one embodiment, the pH of the system is adjusted to between about 8 to about 10, optionally to about 9, optionally using a Bronsted base, such as sodium hydroxide, optionally as an aqueous solution. In one embodiment, the mixture is further stirred at a temperature between about 70° C. to about 90° C., for a period of time of between about 30 min to about 2 hours.

In one embodiment, the mixture as obtained hereinabove is subsequently gradually allowed to cool to about 25° C.

In one embodiment, a further cross-linker, optionally a polyfunctional isocyanate, may be added in step a) and/or in step b). In one embodiment, trimesoyl chloride and/or a polyfunctional isocyanate is added in step a). In one embodiment, glutaraldehyde is added in step b). In one embodiment, a polyfunctional isocyanate is added in step a).

The polyfunctional isocyanate is as defined hereinabove.

### Consumer Product

The present invention also relates to a consumer product comprising a microcapsule composition as described hereinabove. The consumer product may be selected from the group consisting of household (home) care, personal care, fabric care and pet care products.

Suitable home care products include hard surface cleaners, heavy duty detergents and detergent powders, air care compositions.

Suitable personal care products include cleansing compositions (such as shampoos, bath and shower gels, liquid soaps, soap bars), conditioning compositions (such as hair care conditioners), bath and shower lotions, oral care compositions, deodorant compositions, antiperspirant compositions, skin care products.

In one embodiment, the consumer product is a deodorant, in the form of roll-on or a spray.

Suitable fabric care compositions include laundry care detergents, laundry care conditioners, fabric refreshers, scent boosters. In one embodiment, the consumer product is a liquid laundry detergent or a liquid laundry softener or conditioner. In one embodiment, the liquid detergent is a single unit dose liquid detergent.

The microcapsule composition of the present invention, presented in the form of a slurry of microcapsules suspended in an aqueous suspending medium may be incorporated as such in a consumer product base or it may be incorporated in dry powder form.

Drying of a slurry of microcapsules is conventional, and may be carried out according techniques known in the art, such as spray-drying, evaporation, lyophilization or use of a desiccant. Typically, as is conventional in the art, dried microcapsules will be dispersed or suspended in a suitable powder, such as powdered silica, which can act as a bulking agent or flow aid. Such suitable powder may be added to the encapsulated composition before, during or after the drying step.

In particular, the drying process may be accompanied by an additional encapsulation process, wherein an additional functional material is entrapped in an additional encapsulating material. For example, the slurry to be dried may comprise, additionally to the core-shell microcapsules obtained in the process according to the present invention, at least one non-encapsulated functional material and at least one water-soluble encapsulating material, so that the functional material, that is not encapsulated in the core-shell microcapsule, is entrapped in the water-soluble encapsulating material during drying. Typically, the at least one water-soluble encapsulating material comprises at least one hydrocolloid, such as starch octenyl succinate and gum acacia. The hydrocolloid promotes and stabilizes the dispersion of the non-encapsulated material in the aqueous phase of the slurry, so that, upon drying, a matrix is formed around or coexisting with the core-shell microcapsules.

Yet another aspect of the present invention relates to the use of a microcapsule composition as described hereinabove to improve the perception or enhance the performance of the benefit agent in a consumer product.

The present invention is further illustrated by means of the following non-limiting examples.

The microscopic images were recorded with an Olympus BX51 Microscope.

The SEM images were recorded with a Jeol JSM_6010PLUS/LV Scanning electron microscope.

Dv(50) represents the maximum particle diameter below which 50% of the sample volume exists, also known as the median particle size by volume. It is also known as the Malvern volume weighted particle size distribution, measured using light scattering techniques.

The solid content of the slurry has been measured by using a Halogen Moisture Analyzer Mettler Toledo instrument. The solid content, expressed as weight percentage of the initial slurry deposited on the balance may be taken at the point where the drying-induced rate of weight change has dropped below 0.1%/min.

The encapsulation efficacy, i.e. the percentage of the theoretical fragrance that was encapsulated, is given by dividing the measured solid content by theoretical solid content based on the initial amount of encapsulated material and benefit agent involved in the process.

TABLE 1
shows the composition of the fragrance used in the examples

| Fragrance ingredient | Weight (g) |
|---|---|
| AMBRETTOLIDE | 5 |
| DAMASCENONE GIV | 5 |
| DAMASCONE DELTA | 10 |
| FLOROCYCLENE | 300 |
| FRUITATE | 10 |
| HEDIONE | 80 |
| IONONE BETA | 150 |
| ISOJASMONE T | 5 |
| LEMONILE | 10 |
| NECTARYL | 115 |
| OCTINE CARBONATE | 5 |
| METHYLE | 100 |
| PEONILE | 50 |
| PETALIA | 150 |
| SALICYLATE AMYLE | 5 |
| SPIROGALBANONE PURE | |

The performance of the microcapsules was measured by the intensity of the fragrance release during the use experience, such as the pre-rub and post-rub phases in a laundry experience.

The pre-rub phase is the phase when the microcapsules have been deposited on the fabric, e.g., after a fabric consumer product containing microcapsules has been used during the wash cycle. The post-rub phase is after the microcapsules have been deposited and the microcapsules are broken by friction or other similar mechanisms.

### Example 1: (Comparative) Emulsification of Pea Protein with Fragrance

In a 100 g reactor equipped with a mechanical overhead stirrer 3 g of pea protein was mixed with 97.5 g of demineralized water under mild stirring and the mixture was stirred over about 12 h at ambient temperature. The aqueous dispersion was centrifuged and 70 g of the supernatant (containing the soluble part of the protein, 2 g) was used subsequently. The stirring was increased at 700 rpm and 30 g of fragrance was added to this mixture. The temperature was then increased to 80° C. for 5 h 30 min. The heating was stopped and the reaction mixture was allowed to cool down gradually to room temperature.

FIG. 1 shows a microscopic image of the composition, showing only formation of a weak and very elastic membrane around fragrance droplets.

### Example 2: Synthesis of Microcapsules Using Pea Protein and DHA (Dihydroxyacetone) Cross-Linker

In a 100 g reactor equipped with a mechanical overhead stirrer 3 g of pea protein was mixed with 97.5 g of demineralized water. 4.5 g of dihydroxyacetone was added under mild stirring and the mixture was stirred over about 12 h at ambient temperature. The aqueous dispersion was centrifuged and 70 g of the supernatant (containing the soluble part of the protein, 2 g) was used subsequently. 0.2 g of pectin (Roeper APA 104 high methoxy, powder format) was added to help the emulsification step and the mixture was left under stirring for 1 hour. The stirring was increased at 700 rpm and 30 g of fragrance was added to this mixture. The temperature was then increased to 80° C. for 5 h 30 min. The heating was stopped and the reaction mixture was allowed to cool gradually to room temperature.

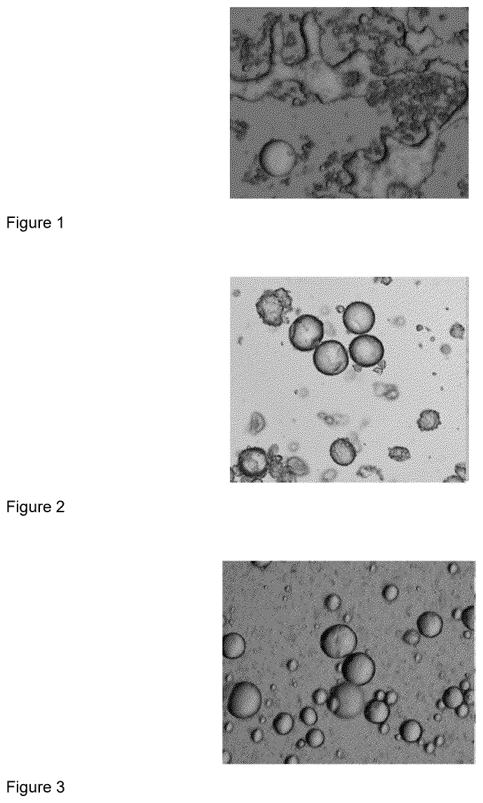

FIG. 2 shows a microscopic image of the composition, showing formation of a mechanically rigid membrane of DHA-cross-linked pea protein coacervate around fragrance droplets. The size of the microcapsules is between about 5 m to about 50 m.

### Example 3: (Comparative) Synthesis of Microcapsules Using Canola Protein

In a 100 g reactor equipped with a mechanical overhead stirrer 2 g of canola protein was mixed with 68 g of demineralized water under mild stirring and the mixture was stirred over about 12 h at ambient temperature. The stirring was increased at 1000 rpm and 30 g of fragrance was added to this mixture. The pH was increased to 9 with an aqueous solution of sodium hydroxide then the temperature was increased to 80° C. for 6 h. The heating was stopped and the reaction mixture was allowed to cool gradually to room temperature.

FIG. 3 shows a microscopic image of the composition, showing formation of an elastic membrane of canola protein coacervate around fragrance droplets.

### Example 4: Synthesis of Microcapsules Using Canola Protein and DHA Cross-Linker

In a 100 g reactor equipped with a mechanical overhead stirrer 2 g of canola protein was mixed with 68 g of demineralized water then 3 g of dihydroxyacetone was added under mild stirring and the mixture was stirred over about 12 h at ambient temperature. The stirring was increased at 1000 rpm and 30 g of fragrance was added to this mixture. The pH was increased to 9 with an aqueous solution of sodium hydroxide then the temperature was increased to 80° C. for 6 h. The heating was stopped and the reaction mixture was allowed to cool gradually to room temperature.

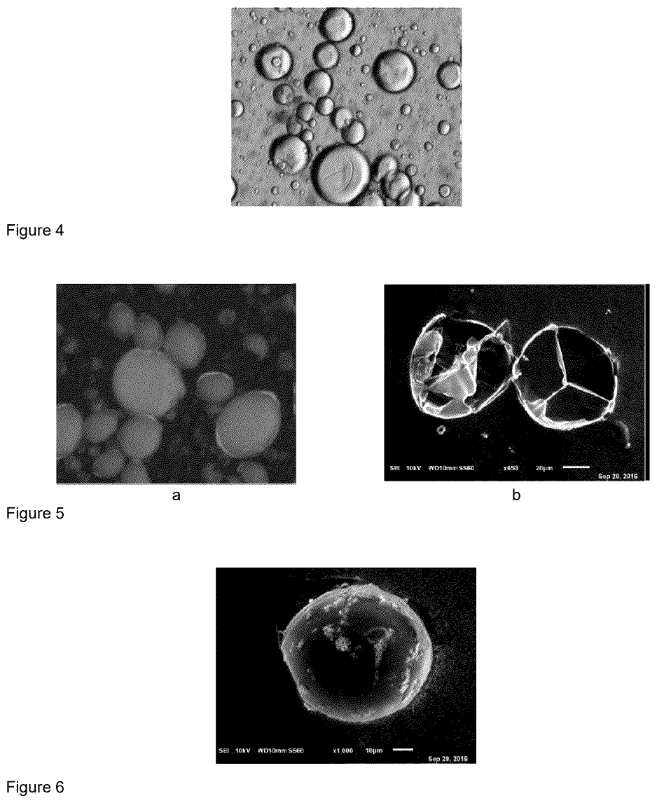

FIG. 4 shows a microscopic image of the composition, showing formation of a rigid membrane of DHA-cross-linked canola protein coacervate around fragrance droplets.

### Example 5: Synthesis of Microcapsules Using Canola Protein, DHA Cross-Linker and Arginine

In a 100 g reactor equipped with a mechanical overhead stirrer 2 g of canola protein was mixed with 68 g of demineralized water then 3 g of dihydroxyacetone was added under mild stirring and the mixture was stirred over about 12 h at ambient temperature. The stirring was increased at 1000 rpm and 30 g of fragrance was added to this mixture. The pH was increased to 9 with an aqueous solution of sodium hydroxide then the temperature was increased to 80° C. for 6 h. Subsequently, 0.5 g of arginine were added to the mixture, the pH was adjusted to 9 and the temperature was kept at 80° C. for 1 hour. The heating was stopped and the reaction mixture was allowed to cool gradually to room temperature.

FIG. 5a shows a microscopic image of the composition, showing formation of a rigid membrane of DHA-cross-linked canola protein coacervate, further cross-linked with arginine, around fragrance droplets.

FIG. 5b shows a SEM image of the composition under high vacuum.

### Example 6: Synthesis of Microcapsules Using Canola Protein, DHA Cross-Linker, Arginine and Isophthalic Dihyrazide

### Example 6.1

In a 100 g reactor equipped with a mechanical overhead stirrer 2 g of canola protein was mixed with 68 g of demineralized water then 3 g of dihydroxyacetone was added under mild stirring and the mixture was stirred over about 12 h at ambient temperature. The stirring was increased at 1000 rpm and 30 g of fragrance was added to this mixture. The pH was increased to 9 with an aqueous solution of sodium hydroxide then the temperature was increased to 80° C. for 6 h. Subsequently, 0.5 g of arginine was added to the mixture, the pH was adjusted to 9 and the temperature was kept at 80° C. for 1 hour. The heating was stopped and when the mixture reached 70° C., 0.5 g of isophthalic dihyrazide was added, the pH was adjusted to 9. The heating was stopped and the reaction mixture was allowed to cool gradually to room temperature.

FIG. 6 illustrates a SEM image of the composition, showing formation of a rigid membrane of DHA-cross-linked canola protein coacervate, further cross-linked with arginine and isophthalic dihyrazide, around fragrance droplets.

The microcapsules showed biodegradability of 60% after 60 days, assessed according to OECD Method 301 F.

### Example 6.2

In a 100 g reactor equipped with a mechanical overhead stirrer 2 g of canola protein was mixed with 30 g of fragrance and the mixture was stirred (400 rpm) over about 12 h at ambient temperature. The stirring was increased at 1100 rpm and 65 g of demineralized water is further added slowly to the oil phase. Subsequently, 3 g of dihydroxyacetone was added, the pH was increased to 9 with an aqueous solution of sodium hydroxide then the temperature was increased to 80° C. for 6 h. Subsequently, 0.5 g of arginine was added to the mixture, the pH was adjusted to 9 and the temperature maintained at 80° C. for 1 hour. 0.5 g of isophthalic dihyrazide was then added, the pH was adjusted to 9 and the reaction mixture was allowed to cool gradually to room temperature.

### Example 7: Encapsulation Efficiency

The encapsulation yield of a core-shell microcapsule is calculated as the ratio of measured solid residue over the sum of all non-volatile ingredients and fragrance employed in the preparation.

TABLE 2
shows the encapsulation yield calculated for the microcapsules
prepared in examples 2-6.

| | | Encapsulation |
|---|---|---|
| Example | Membrane components | yield (%) |
| 1 | Pea protein coacervate | n/a |
| 2 | Pea protein coacervate + DHA | 20 |
| 3 | Canola protein coacervate | 15 |
| 4 | Canola protein coacervate + DHA | 30 |
| 5 | Canola protein coacervate + DHA + arginine | 91 |
| 6 | Canola protein coacervate + DHA + | 100 |
| | arginine + isophthalic dihyrazide | |

It can be observed that only when the DHA cross-linker is employed (Examples 2 and 4-6) encapsulation is observed. When only pea protein was employed (Example 1), the membrane was weak and elastic, not permitting any meaningful fragrance encapsulation. Employing only canola protein (Example 3) led to an improvement in the aspect of the membrane, however the encapsulation efficiency was very low.

### Example 8: Olfactive Performance in Deodorant Application

The microcapsule composition as prepared in Example 6.1 subsequently underwent spray-drying, after which the spray-dried composition was incorporated in two types of deodorant consumer product (spray and roll-on) at a level of 1 wt. % (dry) capsules in the deodorant product.

TABLE 3
shows the composition of the spray and
roll-on deodorant products

| | |
|---|---|
| Ingredient | Weight % |

spray

| | |
|---|---|
| Aluminium chlorohydrate | 10.00 |
| cyclomethicone | 12.00 |
| Disteardimonium hectorite | 0.80 |
| Spray-dried microcapsules | 1.00 |
| Isobutene/propane | 76.20 |

Roll-on

| | |
|---|---|
| Hydroxyethyl cellulose | 0.30 |
| Aluminium chlorohydrate | 40.00 |
| PPG-26 Buteth 26 and PEG-40 | 1.00 |
| Hydrogenated Castor Oil | |
| Spray-dried microcapsules | 1.00 |
| water | 57.70 |

The olfactive performance of the deodorant product was evaluated on a scale of between 0 and 5 (0=no olfactive performance, 5=very high olfactive performance) over time (up to 3 months) at 37° C. The results are shown in Table 4.

TABLE 4
Olfactive performance in roll-on and spray
deodorant applications (at 37° C.)

| | Roll-on | | | Spray | | |
|---|---|---|---|---|---|---|
| | 14 | 2 | 3 | 7 | 2 | 3 |
| | days | months | months | days | months | months |
| Pre rub | 0 | 0 | 0 | 2.5 | 2 | 1.25 |
| Post rub | 3 | 3 | 3 | 3.5 | 3 | 2.25 |

It can be observed that both types of deodorant maintained a very good performance over a period of at least 3 months at 37° C., especially in terms of post-rub performance.

### Example 9: Synthesis of Microcapsules Using Canola Protein, DHA Cross-Linker, Arginine, Isophthalic Dihyrazide and Isocyanate

### Example 9.1

In a 100 g reactor equipped with a mechanical overhead stirrer 2 g of canola protein was mixed with 65 g of demineralized water then 3 g of dihydroxyacetone was added under mild stirring and the mixture was stirred over about 12 h at ambient temperature. The stirring was increased at 1400 rpm and a mixture of 30 g of fragrance and 0.5 g of Takenate D110N was added to the aqueous phase. The pH was increased to 9 with an aqueous solution of sodium hydroxide then the temperature was increased to 80° C. for 6 h. Subsequently, 0.5 g of arginine was added to the mixture, the pH was adjusted to 9 and the temperature was kept at 80° C. for 1 hour. 0.5 g of isophthalic dihyrazide was then added, the pH was adjusted to 9 while the heating was maintained at 80° C. for 1 hour. The heating was stopped and the reaction mixture was allowed to cool gradually to room temperature.

### Example 9.2

In a 100 g reactor equipped with a mechanical overhead stirrer 3 g of canola protein was mixed with 65 g of demineralized water then 3 g of dihydroxyacetone was added under mild stirring and the mixture was stirred over about 12 h at ambient temperature. The stirring was increased at 1400 rpm and a mixture of 30 g of fragrance and 0.5 g of Takenate D110N was added to the aqueous phase. The pH was increased to 9 with an aqueous solution of sodium hydroxide then the temperature was increased to 80° C. for 6 h. Subsequently, 0.5 g of arginine was added to the mixture, the pH was adjusted to 9 and the temperature was kept at 80° C. for 1 hour. 0.5 g of isophthalic dihyrazide was then added, the pH was adjusted to 9 while the heating was maintained at 80° C. for 1 hour. The heating was stopped and the reaction mixture was allowed to cool gradually to room temperature.

### Example 10: Synthesis of Microcapsules Using Pea Protein Hydrolysate, DHA Cross-Linker, Arginine, Isophthalic Dihyrazide and Isocyanate

### Example 10.1

In a 100 g reactor equipped with a mechanical overhead stirrer 2 g of pea protein hydrolysate was mixed with 65 g of demineralized water then 3 g of dihydroxyacetone was added under mild stirring and the mixture was stirred over about 12 h at ambient temperature. The stirring was increased at 1100 rpm and a mixture of 30 g of fragrance and 0.5 g of Takenate D110N was added to the aqueous phase. The pH was increased to 9 with an aqueous solution of sodium hydroxide then the temperature was increased to 80° C. for 6 h. Subsequently, 0.5 g of arginine was added to the mixture, the pH was adjusted to 9 and the temperature was kept at 80° C. for 1 hour. 0.5 g of isophthalic dihyrazide was then added, the pH was adjusted to 9 while the heating was maintained at 80° C. for 1 hour. The heating was stopped and the reaction mixture was allowed to cool gradually to room temperature.

The microcapsules showed biodegradability of 60% after 60 days, assessed according to OECD Method 301 F.

### Example 10.2

In a 100 g reactor equipped with a mechanical overhead stirrer 1 g of pea protein hydrolysate was mixed with 67 g of demineralized water then 3 g of dihydroxyacetone was added under mild stirring and the mixture was stirred over about 30 min at ambient temperature. In a separate beaker a mixture of 2.5 g of pea protein hydrolysate, 30 g of fragrance and 0.5 g of Takenate D110N was stirred for 30 minutes at 50° C. The stirring in the reactor was increased to 1100 rpm and the fragrance mixture was added to the aqueous phase. The pH was increased to 9 with an aqueous solution of sodium hydroxide then the temperature was increased to 80° C. for 6 h. Subsequently, 0.5 g of arginine was added to the mixture, the pH was adjusted to 9 and the temperature maintained at 80° C. for 1 hour. 0.5 g of isophthalic dihyrazide was then added, the pH was adjusted to 9 and the reaction mixture was allowed to cool gradually to room temperature.

### Example 11: Olfactive Performance in Deodorant Application

The microcapsule composition as prepared in Example 9.1 was incorporated into a liquid fabric softener base and a liquid detergent base, at a level of 0.15 wt. % of perfume (0.5 wt % of microcapsules) in the final consumer product. Table 5 shows the composition of the liquid fabric softener and the liquid detergent bases.

TABLE 5

| | |
|---|---|
| Ingredient | Weight % |

Liquid fabric softener

| | |
|---|---|
| Dialkylester Ammonium Methosulfate | 8.9 |
| PEG-40 hydrogenated castor oil | 1.0 |
| Sodium benzoate | 0.3 |
| CaCl₂ | 0.4 |
| Citric acid 20% | 1.9 |
| water | 84.5 |

Liquid detergent

| | |
|---|---|
| Dipropylene glycol | 4.0 |
| Glycerol | 2.8 |
| Sodium Laureth Sulfate | 4.1 |
| Alkyl Benzene Sulfonic Acid | 8.3 |
| Lauric acid | 2.0 |
| Sodium hydroxide | 3.0 |
| alcohol ethoxylate, nonionic surfactant | 6.0 |
| preservative | 0.03 |
| EDTA—H₂Na₂ | 0.2 |
| water | 69.6 |

The olfactive performance of the consumer products was evaluated on a scale of between 0 and 5 (0=no olfactive performance, 5=very high olfactive performance) at 37° C. The results are shown in Table 6.

TABLE 6
Olfactive performance in laundry softener
and laundry detergent applications

| Consumer product | Textile Material | wet | Pre-rub | Post-rub |
|---|---|---|---|---|
| Softener | Cotton | 1 | 2 | 2 |
| Liquid detergent | Cotton | 2.5 | 0.5 | 1 |
| Liquid detergent | Polyester | 3 | 1.5 | 2 |

Both types of consumer product comprising microcapsules according to the present invention showed very good performance on different types of textiles, especially in terms of post-rub performance, with better overall performance observed on polyester textiles.

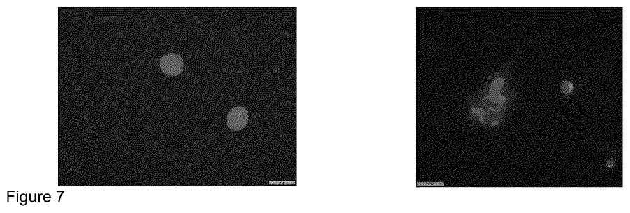

FIG. 7 illustrates a comparison between the microscopic aspect of the microcapsules prepared as in Example 9.1 (left) at 2 weeks after incorporation in a liquid detergent base as described hereinabove and the microscopic aspect of the microcapsules prepared as in Example 6.1 (right) at about 10 min after incorporation in the same liquid detergent base, at the same perfume loading.

1. An encapsulated composition comprising at least one core-shell microcapsule, wherein the at least one core-shell microcapsule comprises a core comprising at least one benefit agent and a shell surrounding the core, wherein the shell comprises a cross-linked-protein coacervate, wherein the cross-linker is dihydroxyacetone.

2. The encapsulated composition according to claim 1, wherein the cross-linked-protein coacervate comprises a coacervate of a cross-linked protein.

3. The encapsulated composition according to claim 1, wherein the protein is an animal-origin protein, a vegetable-origin protein or a bioengineered protein.

4. The encapsulated composition according to claim 1, wherein the protein is selected from the group consisting of canola or rapeseed protein, hemp protein, potato protein, chickpea protein, pea protein hydrolysate, pea protein, pea and rice fermented protein, mungbean protein, sunflower protein, pumpkin protein, carob protein, fava bean protein.

5. The encapsulated composition according to claim 1, wherein the weight ratio between the dihydroxyacetone moieties and the protein moieties in the composition is between about 2:1 to about 1:1.

6. The encapsulated composition according to claim 1, wherein the dihydroxyacetone-cross-linked protein is further cross-linked with a second cross-linker comprising more than two amino functionalities.

7. The encapsulated composition according to claim 6, wherein the second cross-linker comprising more than two amino functionalities is selected from the group consisting of aminoacids with more than two amino functionalities, derivatives or polymers thereof, polyhydrazides and combinations thereof.

8. The encapsulated composition according to claim 6, wherein the second cross-linker comprising more than two amino functionalities is selected from the group consisting of arginine, polyarginine, PCA ethyl cocoyl arginate, lysine, polylysine, histidine, isophthalic dihydrazide and combinations thereof.

9. The encapsulated composition according to claim 1, comprising a further cross-linker selected from the group consisting of trimesoyl chloride (1,3,5-benzenetricarbonyl trichloride), a polyfunctional isocyanate, glutaraldehyde and combinations thereof.

10. The encapsulated composition according to claim 1, wherein the composition comprises between about 0.5 wt % to about 7 wt % dihydroxyacetone moieties with respect to the composition.

11. The encapsulated composition according to claim 1, wherein the benefit agent is biodegradable.

12. A method for preparing an encapsulated composition according to claim 1, the method comprising the steps of:
a) providing an aqueous phase comprising a protein and dihydroxyacetone;
b) providing an oil phase comprising a benefit agent;
c) emulsifying the oil phase provided in step b) in the aqueous phase composition provided in step a);
d) inducing coacervation of the mixture obtained in step c) to obtain core composition droplets surrounded by a polymeric membrane; and
d) optionally, adding to the composition obtained in step c) a second cross-linker comprising more than two amino functionalities to obtain a microcapsule composition;
optionally wherein the oil phase in step b) comprises a further cross-linker.

13. A method for preparing an encapsulated composition according to claim 1, the method comprising the steps of:
a) providing an oil phase comprising a protein and benefit agent;
b) providing an aqueous phase comprising dihydroxyacetone;
c) emulsifying the oil phase provided in step a) in the aqueous phase composition provided in step b);
d) inducing coacervation of the mixture obtained in step c) to obtain core composition droplets surrounded by a polymeric membrane; and
d) optionally, adding to the composition obtained in step c) a second cross-linker comprising more than two amino functionalities to obtain a microcapsule composition;
optionally wherein the oil phase in step a) comprises a further cross-linker.

14. An encapsulated composition obtained by the method according to claim 12.

15. A method of enhancing the performance of a benefit agent in a consumer product, comprising the step of:
providing a core comprising at least one benefit agent encapsulated by a shell surrounding the core, wherein the shell comprises a cross-linked-protein coacervate, wherein the cross-linker is dihydroxyacetone.

16. A consumer product comprising an encapsulated composition according to claim 1.

17. The encapsulated composition according to claim 3, wherein preferably wherein the protein is a vegetable-origin protein selected from: proteins from cereals, proteins from high-protein plants, proteins from oleaginous plants and proteins from tubers.

18. The encapsulated composition according to claim 9, wherein the further cross-linker is an alkylaromatic polyfunctional isocyanate.

19. The encapsulated composition according to claim 1 wherein the composition comprises between about 2.5 wt % to about 3.5 wt % dihydroxyacetone moieties with respect to the composition.

20. The encapsulated composition according to claim 1 wherein the composition comprises about 3.0 wt % dihydroxyacetone moieties with respect to the composition.
